(19) United States
(12) Patent Application Publication
(10) Pub. No.: US 2024/0374543 A1
(43) Pub. Date: Nov. 14, 2024

(54) PHARMACEUTICAL COMPOSITIONS

(71) Applicant: Novast Laboratories, Ltd, Nantong, Jiangsu (CN)

(72) Inventors: Jian Gong, Nantong, Jiangsu (CN); Manman Liu, Nantong, Jiangsu (CN); Chengsong Hu, Nantong, Jiangsu (CN); Yisheng Chen, Nantong, Jiangsu (CN); Guohua Zhang, Nantong, Jiangsu (CN)

(73) Assignee: Novast Laboratories, Ltd, Nantong, Jiangsu (CN)

(21) Appl. No.: 18/532,226

(22) PCT Filed: Dec. 30, 2021
(86) PCT No.: PCT/CN2021/143011

§ 371 Date: Dec. 7, 2023

(30) Foreign Application Priority Data
Jun. 7, 2021 (CN) 202110633007.7

(51) Int. Cl. A61K 31/155; A61K 9/16; A61K 9/20; A61K 9/28; A61K 31/4045; A61K 31/41; A61K 31/4178; A61K 31/4406; A61K 31/554; A61K 31/65

(52) U.S. Cl. CPC A61K 31/155; A61K 9/1611; A61K 9/1617; A61K 9/1635; A61K 9/1652; A61K 9/2009; A61K 9/2013; A61K 9/2027; A61K 9/2054; A61K 9/2866; A61K 31/4045; A61K 31/41; A61K 31/4178; A61K 31/4406; A61K 31/554; A61K 31/65

(57) ABSTRACT

A Pharmaceutical composition for inhibiting N-nitrosodimethylamine (NDMA) formation, and a method. In a pharmaceutical composition containing a selection of metformin, valsartan, irbesartan, losartan, sumatriptan, diltiazem, tetracycline, doxylamine, Chlorpheniramine, carbinoxamine, and/or any salt thereof, a non-antioxidant acidic component is added, causing N-nitrosodimethylamine content within an effective period of the pharmaceutical to be suppressed to lower than an acceptable daily intake limit therefor.

### BACKGROUND

N-Nitrosodimethylamine (NDMA) is an environmental contaminant of N-nitrosamines that commonly exists in the air, water, and foods. It has been discovered that NDMA is hepatotoxic and that exposure to NDMA above acceptable levels and over long periods may increase the risk of cancer. The Food and Drug Administration (FDA) has determined that the acceptable daily intake limit of NDMA is 96 nanogram per day.

It has also been discovered that many pharmaceutical compositions contain potentially harmful amounts of NDMA. NDMA can form during certain drug manufacturing processes, and when using certain ingredients, including ingredients not intended to be in the final product, but which are not adequately purged during manufacturing.

Therefore, there remains a need for compositions and methods for minimizing formation or intake of NDMA.

### SUMMARY

In one aspect, the present disclosure relates to pharmaceutical compositions that inhibit the formation of N-nitrosodimethylamine (NDMA) over time.

In some preferred aspects, the formation of N-nitrosodimethylamine is inhibited to a level that is below the acceptable daily intake limit during the shelf life of drug products comprising one or more active pharmaceutical ingredient(s). In accordance with some forms, the active pharmaceutical ingredients are selected from the group consisting of metformin, valsartan, irbesartan, losartan, sumatriptan, diltiazem, tetracycline, doxylamine, chlorpheniramine, carbinoxamine, and/or any salts thereof.

In certain embodiments, the present disclosure provides pharmaceutical compositions comprising non-antioxidant acidic component.

In another aspect, the present disclosure relates to pharmaceutical compositions having reduced levels of N-nitrosodimethylamine. In accordance with some forms, pharmaceutical compositions of the present disclosure can be administered such that not more than 40 ng of N-nitrosodimethylamine is administered when calculated using the maximum daily dose of the pharmaceutical compound.

In still other aspects, the present disclosure relates to methods for reducing the daily intake of N-nitrosodimethylamine in a patient in need of treatment.

In yet further aspects, the present disclosure relates to methods for inhibiting the formation of N-nitrosodimethylamine in a pharmaceutical product.

Additional aspects and embodiments of the invention, as well as features and advantages thereof, will be apparent from the descriptions herein.

### DETAILED DESCRIPTION

For the purposes of promoting an understanding of the principles of the invention, reference will now be made to certain embodiments and specific language will be used to describe the same. It will nevertheless be understood that no limitation of the scope of the invention is thereby intended, such alterations and further modifications, and such further applications of the principles of the invention as described herein being contemplated as would normally occur to one skilled in the art to which the invention relates. Additionally, in the detailed description below, numerous alternatives are given for various features. It will be understood that each such disclosed alternative, or combinations of such alternatives, can be combined with the more generalized features discussed in the Summary above, or set forth in the embodiments described below to provide additional disclosed embodiments herein.

“N-nitrosodimethylamine”, sometimes referred to as “NDMA” which is used interchangeably within this disclosure and should both be understood to refer to the following chemical structure, Structure 1:

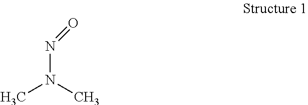

In recent years, NDMA and other N-nitrosamine contaminants have been found in various drug products. Particularly, many drug products of the common antidiabetic drug metformin hydrochloride, H2 antagonist ranitidine hydrochloride, nizatidine, and anti-hypertension drug products of a group of Angiotensin II receptor blocker including valsartan, irbesartan, and losartan potassium, are contaminated by NDMA at above acceptable daily intake limits, which lead to the wide spread withdrawal of the unacceptable drug products from the market by many drug manufacturers. On Apr. 1, 2020, the US Food and Drug Administration (FDA) requested removal of all ranitidine drug products from the market. The FDA and the European Medicines Agency (EMA) require the all drug product manufacturers to evaluate the risk of nitrosamines being present in their products, and to take appropriate risk mitigating measures.

Currently, the root causes for the existence of NDMA in pharmaceutical products are not yet clear, and the levels of NDMA levels in drug products are uncertain. On Apr. 1, 2020, FDA requested all ranitidine products be withdrawn from the US market. As a result, patients could not access to the medicine for treatment at the present time. Large number of lots of sartans and metformin extended release (ER) products were found to contain NDMA above the daily intake limit, and those products were withdrawn from the market. For the US market alone, FDA information indicates that many companies have recalled at least a total of 128 lots of metformin extended release tablets during a short period of time from June 2020 to November 2020 due to the NDMA exceeding the daily intake limit in the drug product. Drug product manufacturers are now asked by FDA to test each batch of at-risk products, and to release the products onto the U.S. market only if testing shows NDMA is not above the acceptable intake limit.

In certain embodiments of the present invention, some active pharmaceutical ingredients may be used. For example, metformin, valsartan, irbesartan, losartan, sumatriptan, diltiazem, tetracycline, doxylamine, chlorpheniramine, carbinoxamine and/or any salts thereof may be used.

Active pharmaceutical ingredients may be combined with other substances in some embodiments of the present invention. In preferred embodiments, such other substances are preferably inert and/or do not affect the efficacy of an active pharmaceutical ingredient. In some embodiments, a binder, a filler, a lubricant, and/or a rate-controlling ingredient for dissolution of an active pharmaceutical ingredient may be utilized.

A binder may be selected from starch, gelatin, zein, guar gum, poloxamer, polyethylene oxide, polyvinylpyrrolidone, ethylcellulose, methylcellulose, hydroxypropyl cellulose, hydroxypropylmethyl cellulose, hydroxyethyl cellulose, microcrystalline cellulose, sorbitol, glucose, dextrose, sucrose, or any combinations thereof.

A filler may be selected from starches, lactose, sucrose, glucose, dextrose, mannitol, sorbitol, microcrystalline celluloses, calcium carbonate, calcium sulfate, or any combinations thereof.

A rate-controlling ingredient for dissolution of an active pharmaceutical ingredient may be selected from shellac, cellulosic polymers, polysaccharides, polyacrylic acids, methacrylic acid copolymers, proteins, polyethylene oxides, poloxamers, polyvinyl pyrrolidone, polyvinyl alcohols, polyvinyl acetates, fatty acids, alcohol esters, waxes, or any combinations thereof.

A lubricant can be selected from polyethylene glycols, glycerol esters, hydrogenated vegetable oils, mineral oils, fatty acids, metal stearates, starch, sodium stearyl fumarate, sodium lauryl sulfate, sodium oleate, talc, boric acid, sodium benzoate, sodium chloride, or any combinations thereof.

As disclosed herein, the present disclosure includes pharmaceutical compositions having a non-antioxidant acidic ingredient. Acidic ingredients in this invention include any ingredient that can result in a solution acidity (pH) of not higher than pH 5 when the ingredient is mixed at a concentration of not more than 5% (w/w) in water at room temperature. Suitable acidic ingredients for use in the present disclosured compositions include, but are not limited to: hydrochloric acid, sulfuric acid, phosphoric acid, boric acid, benzene sulphonic acid, acetic acid, adipic acid, alginic acid, benzoic acid, carbomer, citric acid, fumaric acid, lactic acid, maleic acid, malic acid, tartaric acid, succinic acid, formic acid, oxalic acid, oleic acid, sorbic acid, pentetic acid, polycarbophil, salicylic acid, acetyl salicylic acid, glycine hydrochloride, L-cystine dihydrochloride, glutamic acid, aspartic acid, disodium edetate, edetic acid, salts of citrate monobasic, salts of phosphate monobasic, enteric polymers, or any combinations thereof.

In order to promote a further understanding of the present invention and its various embodiments, the following specific examples are provided. It will be understood that these examples are illustrative and not limiting of the invention.

### Example 1

### Control Commercial Pharmaceutical Composition

A commercial product of metformin hydrochloride tablets was manufactured using the formula list in Table 1 and stored at room temperature for 14 months and was tested for presence of NDMA.

TABLE 1
Compositions and NDMA Level in a Commercial Metformin
Hydrochloride Extended Release Product

| | Formula 1 |
|---|---|
| Ingredients | (mg/tablet) |
| Metformin Hydrochloride | 500 |
| Celluloses | 122.5 |
| Povidone | 25.5 |
| Magnesium Stearate | 1.5 |
| Cellulose Coating | 38 |
| NDMA (ng/g Metformin HCl) | 24 |

The active and inactive ingredients used for the above product were substantially free of NDMA, and the product was manufactured with strict controls in a clean environment with clean equipment. The exact sources or mechanisms of the NDMA contamination during the manufacturing and storage of drug products is not clear.

These data show that NDMA exists in the commercial product at a level of 24 ng per gram of metformin hydrochloride. The result can be used to calculate that the daily intake of NDMA by maximum daily dosing of 2 g of metformin hydrochloride per labeling of the product is 48 ng/day. Although the result is below the daily intake limit, it is consistent with the widely known fact that many commercial products of metformin hydrochloride extended release products are contaminated by NDMA.

### Example 2

### Improved Pharmaceutical Compositions

Different acidic ingredients and alkaline agent were used for preparation of metformin compositions as listed in Table 2. The compositions were granulated by a wet high shear granulation process using water as a granulation fluid. The resulting granules were then tested for NDMA. Results are listed in Table 2. The results surprisingly show that formation of NDMA in the compositions was inhibited in composition containing acidic ingredients, while the use of an alkaline agent accelerated the formation of NDMA.

TABLE 2
Formulations of Metformin HCl Extended Release Granules

| | mg/1000 mg Metformin HCl | | | | | | |
|---|---|---|---|---|---|---|---|
| | Formula | Formula | Formula | Formula | Formula | Formula | Formula |
| Ingredients | 2 | 3 | 4 | 5 | 6 | 7 | 8 |
| Metformin | 1000 | 1000 | 1000 | 1000 | 1000 | 1000 | 1000 |
| Hydrochloride | | | | | | | |
| Celluloses | 180 | 180 | 180 | 180 | 180 | 180 | 180 |
| Povidone | 51 | 51 | 51 | 51 | 51 | 51 | 51 |
| Purified water | q.s. | q.s. | q.s. | q.s. | q.s. | q.s. | q.s. |
| Disodium edetate | / | 3 | 10 | / | / | / | / |
| Fumaric acid | / | / | / | 5 | / | / | / |
| Hydrochloric acid | / | / | / | / | 5 | 18.5 | / |
| Sodium Hydroxide | / | / | | / | / | / | 5 |
| NDMA (ng/g- | 7.6 | 0 | 0 | 0 | 0 | 0 | 26.2 |
| Metformin HCl) | | | | | | | |

### Example 3

### Improved Pharmaceutical Compositions

Based on the results of Example 2, different acidic ingredients, including fumaric acid, tartaric acid, hydrochloride, and disodium edetate were used as NDMA inhibitors in the compositions of metformin hydrochloride extended release tablets. The tablets with and without acidic ingredient were manufactured according to the compositions listed in Table 3 via high shear wet granulation, drying, milling, blending, compression, and tablet coating steps. Cellulose coating was used to modify the API release in the product. The tablets were packaged in high-density polyethylene (HDPE) bottles with heat induction seal and stored at accelerated conditions of 40° C./75% relative humidity for stability study over 3 months.

NDMA results for stability samples summarized in Table 3 show that each of the compositions including acidic ingredients effectively reduced the formation of NDMA in the compositions as compared with the tablets without acidic ingredients.

TABLE 3
Compositions and NDMA Results (Stored at 40° C./75% RH) of Metformin
HCl Extended Release Tablets with and without Acidic Ingredient

| | mg/tablet | | | | | | | | |
|---|---|---|---|---|---|---|---|---|---|
| | Formula | Formula | Formula | Formula | Formula | Formula | Formula | Formula | Formula |
| Ingredients | 9 | 10 | 11 | 12 | 13 | 14 | 15 | 16 | 17 |
| Metformin | 1000 | 1000 | 1000 | 1000 | 1000 | 1000 | 1000 | 1000 | 1000 |
| Hydrochloride | | | | | | | | | |
| Celluloses | 180 | 180 | 180 | 180 | 180 | 180 | 180 | 180 | 180 |
| Povidone | 51 | 51 | 51 | 51 | 51 | 51 | 51 | 51 | 51 |
| Purified water | q.s | q.s | q.s | q.s | q.s | q.s | q.s | q.s | q.s |
| Disodium | / | 5 | 10 | / | / | / | / | / | / |
| edetate | | | | | | | | | |
| Fumaric acid | / | / | / | 5 | 10 | / | / | / | / |
| Tartaric acid | / | / | / | / | / | 5 | 10 | / | / |
| Hydrochloric | / | / | / | / | / | / | / | 5 | 10 |
| acid | | | | | | | | | |
| Magnesium | 3 | 3 | 3 | 3 | 3 | 3 | 3 | 3 | 3 |
| Stearate | | | | | | | | | |
| Cellulose | 51 | 51 | 51 | 51 | 51 | 51 | 51 | 51 | 51 |
| Coating | | | | | | | | | |

NDMA Results (ng/g of Metformin HCl) at 40 C./75% RH

| | | | | | | | | | |
|---|---|---|---|---|---|---|---|---|---|
| Initial | 0 | 0 | 0 | 3.1 | 0 | 0 | 0 | 0 | 0 |
| 1 Month | 5.4 | 6.3 | 6.1 | 0 | 0 | 0 | 0 | 0 | 0 |
| 2 Months | 10.5 | 4.2 | 4.9 | 0 | 0 | 0 | 0 | 0 | 0 |
| 3 Months | 14.3 | 2.9 | 3.3 | 0 | 0 | 0 | 0 | 0 | 0 |

The uses of the terms “a” and “an” and “the” and similar references in the context of describing the invention (especially in the context of the following claims) are to be construed to cover both the singular and the plural unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

While the invention has been illustrated and described in detail in the drawings and the foregoing description, the same is to be considered as illustrative and not restrictive in character, it being understood that only the preferred embodiment has been shown and described and that all changes and modifications that come within the spirit of the invention are desired to be protected. In addition, all references cited herein are indicative of the level of skill in the art and are hereby incorporated by reference in their entirety.

### Embodiments

The following is a list of some embodiments in the present disclosure. It should be understood that the list is not restrictive and that the individual features or combinations of features (such as, 2, 3 or 4 features) as described in the specific examples above may be combined with the embodiments listed below to provide the annex embodiments disclosed herein.

1. A pharmaceutical composition comprising:
an active pharmaceutical ingredient, wherein said active pharmaceutical ingredient is selected from the group consisting of metformin, valsartan, irbesartan, losartan, sumatriptan, diltiazem, tetracycline, doxylamine, chlorpheniramine, carbinoxamine, and/or any salts thereof;
a non-antioxidant acidic ingredient; and
an excipient;
   2. The pharmaceutical composition of embodiment 1, wherein the said acidic ingredient is selected from the group comprising hydrochloric acid, sulfuric acid, phosphoric acid, boric acid, benzene sulphonic acid, acetic acid, adipic acid, alginic acid, benzoic acid, carbomer, citric acid, fumaric acid, lactic acid, maleic acid, malic acid, tartaric acid, succinic acid, formic acid, oxalic acid, oleic acid, sorbic acid, pentetic acid, polycarbophil, salicylic acid, acetyl salicylic acid, glycine hydrochloride, L-cystine dihydrochloride, glutamic acid, aspartic acid, disodium edetate, edetic acid, salts of citrate monobasic, salts of phosphate monobasic, enteric polymers, or any combinations thereof.
   3. The pharmaceutical composition of embodiment 1 or 2, wherein the composition comprises 1 mg to 100 mg of said acidic ingredient per gram of said active pharmaceutical ingredient.
   4. The pharmaceutical composition of embodiment 1-3, wherein said composition contains not more than 40 ng of N-nitrosodimethylamine when calculated using the maximum daily dose per the respective drug label.
   5. The pharmaceutical composition of embodiment 1, wherein said excipient is selected from the group comprising a binder, a filler, a lubricant, or a rate-controlling ingredient for dissolution of said active pharmaceutical ingredients.
   6. The pharmaceutical composition of embodiment 5, wherein said binder is selected from the group comprising starch, gelatin, zein, guar gum, poloxamer, polyethylene oxide, polyvinylpyrrolidone, ethylcellulose, methylcellulose, hydroxypropyl cellulose, hydroxypropylmethyl cellulose, hydroxyethyl cellulose, microcrystalline cellulose, sorbitol, glucose, dextrose, sucrose, or any combinations thereof.
   7. The pharmaceutical composition of embodiment 5, where said filler is selected from the group comprising starch, lactose, sucrose, glucose, dextrose, mannitol, sorbitol, microcrystalline celluloses, calcium carbonate, calcium sulfate, or any combinations thereof.
   8. The pharmaceutical composition of embodiment 5, wherein said lubricant is selected from the group comprising polyethylene glycols, glycerol esters, hydrogenated vegetable oils, mineral oils, fatty acids, metal stearates, starch, sodium stearyl fumarate, sodium lauryl sulfate, sodium oleate, talc, boric acid, sodium benzoate, sodium chloride, or any combinations thereof.
   9. The pharmaceutical composition of embodiment 5, wherein said rate-controlling ingredient for dissolution of said active pharmaceutical ingredients is selected from the group comprising shellac, cellulosic polymers, polysaccharides, polyacrylic acids, methacrylic acid copolymers, proteins, polyethylene oxides, poloxamers, polyvinyl pyrrolidone, polyvinyl alcohols, polyvinyl acetates, fatty acids, alcohol esters, waxes, or any combinations thereof.
   10. A method for reducing the daily intake of N-nitrosodimethylamine in a patient in need of treatment, the method comprising:
adding a non-antioxidant acidic ingredient as an inhibitor of N-nitrosodimethylamine to a composition comprising an active pharmaceutical ingredient to form a first composition of matter; and administering said first composition of matter to said patient in need of treatment.
   11. The method of embodiment 10, wherein said non-antioxidant acidic ingredient is selected from the group comprising hydrochloric acid, sulfuric acid, phosphoric acid, boric acid, benzene sulphonic acid, acetic acid, adipic acid, alginic acid, benzoic acid, carbomer, citric acid, fumaric acid, lactic acid, maleic acid, malic acid, tartaric acid, succinic acid, formic acid, oxalic acid, oleic acid, sorbic acid, pentetic acid, polycarbophil, salicylic acid, acetyl salicylic acid, glycine hydrochloride, L-cystine dihydrochloride, glutamic acid, aspartic acid, disodium edetate, edetic acid, salts of citrate monobasic, salts of phosphate monobasic, enteric polymers, or any combinations thereof.
   12. The method of embodiment 10, wherein said active pharmaceutical ingredient is selected from the group consisting of metformin, valsartan, irbesartan, losartan, sumatriptan, diltiazem, tetracycline, doxylamine, chlorpheniramine, carbinoxamine, and/or any salts thereof.
   13. The method of embodiment 10, wherein said daily intake of N-nitrosodimethylamine administered to said patient in need of treatment is less than 40 ng per day when the composition is administered at the maximum daily dose per the respective drug label.
   14. A method for inhibiting the formation of N-nitrosodimethylamine in a pharmaceutical composition, the method comprising the acts of:
providing an active pharmaceutical ingredient, wherein said active pharmaceutical ingredient is selected from the group consisting of metformin, valsartan, irbesartan, losartan, sumatriptan, diltiazem, tetracycline, doxylamine, chlorpheniramine, carbinoxamine, and/or any salts thereof; and
adding non-antioxidant acidic ingredient as an inhibitor of N-nitrosodimethylamine to said active pharmaceutical ingredient to form a pharmaceutical composition.
   15. The method of embodiment 14, wherein said non-antioxidant acidic ingredient is selected from the group comprising hydrochloric acid, sulfuric acid, phosphoric acid, boric acid, benzene sulphonic acid, acetic acid, adipic acid, alginic acid, benzoic acid, carbomer, citric acid, fumaric acid, lactic acid, maleic acid, malic acid, tartaric acid, succinic acid, formic acid, oxalic acid, oleic acid, sorbic acid, pentetic acid, polycarbophil, salicylic acid, acetyl salicylic acid, glycine hydrochloride, L-cystine dihydrochloride, glutamic acid, aspartic acid, disodium edetate, edetic acid, salts of citrate monobasic, salts of phosphate monobasic, enteric polymers, or any combinations thereof.
   16. The method of embodiment 14 or 15, further comprising the act of adding an excipient to form a pharmaceutical composition.
   17. The method of embodiment 16, wherein said excipient is selected from the group comprising a binder, a filler, a lubricant, or a rate-controlling ingredient for dissolution of said active pharmaceutical ingredients.
   18. The method of any one of embodiment 14-17, wherein said pharmaceutical composition comprises less than 40 ng N-nitrosodimethylamine when the pharmaceutical composition is administered at the maximum daily dose per the respective drug label.

Although the present invention has been illustrated and described in detail in the preceding description, these illustrations and descriptions are considered to be illustrative and not restrictive, and it should be understood that only the preferred embodiment is shown and described, and it is desirable to protect all changes, equivalents and modifications that fall within the spirit of the present invention as defined by the following claims. All publications, patents and patent applications referenced in this specification are incorporated herein by reference as if each individual publication, patent or patent application were specifically and separately indicated by reference incorporated herein and set forth in its entirety herein.

1. A pharmaceutical composition comprising:
an active pharmaceutical ingredient, wherein said active pharmaceutical ingredient is selected from the group consisting of metformin, valsartan, irbesartan, losartan, sumatriptan, diltiazem, tetracycline, doxylamine, chlorpheniramine, carbinoxamine, and/or any salts thereof;
a non-antioxidant acidic ingredient; and
an excipient.

2. The pharmaceutical composition of claim 1, wherein the said acidic ingredient is selected from the group comprising hydrochloric acid, sulfuric acid, phosphoric acid, boric acid, benzene sulphonic acid, acetic acid, adipic acid, alginic acid, benzoic acid, carbomer, citric acid, fumaric acid, lactic acid, maleic acid, malic acid, tartaric acid, succinic acid, formic acid, oxalic acid, oleic acid, sorbic acid, pentetic acid, polycarbophil, salicylic acid, acetyl salicylic acid, glycine hydrochloride, L-cystine dihydrochloride, glutamic acid, aspartic acid, disodium edetate, edetic acid, salts of citrate monobasic, salts of phosphate monobasic, enteric polymers, or any combinations thereof.

3. The pharmaceutical composition of claim 1, wherein the composition comprises 1 mg to 100 mg of said acidic ingredient per gram of said active pharmaceutical ingredient.

4. The pharmaceutical composition of claim 1, wherein said composition contains not more than 40 ng of N-nitrosodimethylamine when calculated using the maximum daily dose per the respective drug label.

5. The pharmaceutical composition of claim 1, wherein said excipient is selected from the group comprising a binder, a filler, a lubricant, or a rate-controlling ingredient for dissolution of said active pharmaceutical ingredients.

6. The pharmaceutical composition of claim 5, wherein said binder is selected from the group comprising starch, gelatin, zein, guar gum, poloxamer, polyethylene oxide, polyvinylpyrrolidone, ethylcellulose, methylcellulose, hydroxypropyl cellulose, hydroxypropylmethyl cellulose, hydroxyethyl cellulose, microcrystalline cellulose, sorbitol, glucose, dextrose, sucrose, or any combinations thereof.

7. The pharmaceutical composition of claim 5, where said filler is selected from the group comprising starch, lactose, sucrose, glucose, dextrose, mannitol, sorbitol, microcrystalline celluloses, calcium carbonate, calcium sulfate, or any combinations thereof.

8. The pharmaceutical composition of claim 5, wherein said lubricant is selected from the group comprising polyethylene glycols, glycerol esters, hydrogenated vegetable oils, mineral oils, fatty acids, metal stearates, starch, sodium stearyl fumarate, sodium lauryl sulfate, sodium oleate, talc, boric acid, sodium benzoate, sodium chloride, or any combinations thereof.

9. The pharmaceutical composition of claim 5, wherein said rate-controlling ingredient for dissolution of said active pharmaceutical ingredients is selected from the group comprising shellac, cellulosic polymers, polysaccharides, polyacrylic acids, methacrylic acid copolymers, proteins, polyethylene oxides, poloxamers, polyvinyl pyrrolidone, polyvinyl alcohols, polyvinyl acetates, fatty acids, alcohol esters, waxes, or any combinations thereof.

10. A method for reducing the daily intake of N-nitrosodimethylamine in a patient in need of treatment, the method comprising:
adding a non-antioxidant acidic ingredient as an inhibitor of N-nitrosodimethylamine to a composition comprising an active pharmaceutical ingredient to form a first composition of matter; and
administering said first composition of matter to said patient in need of treatment.

11. The method of claim 10, wherein said non-antioxidant acidic ingredient is selected from the group comprising hydrochloric acid, sulfuric acid, phosphoric acid, boric acid, benzene sulphonic acid, acetic acid, adipic acid, alginic acid, benzoic acid, carbomer, citric acid, fumaric acid, lactic acid, maleic acid, malic acid, tartaric acid, succinic acid, formic acid, oxalic acid, oleic acid, sorbic acid, pentetic acid, polycarbophil, salicylic acid, acetyl salicylic acid, glycine hydrochloride, L-cystine dihydrochloride, glutamic acid, aspartic acid, disodium edetate, edetic acid, salts of citrate monobasic, salts of phosphate monobasic, enteric polymers, or any combinations thereof.

12. The method of claim 10, wherein said active pharmaceutical ingredient is selected from the group consisting of metformin, valsartan, irbesartan, losartan, sumatriptan, diltiazem, tetracycline, doxylamine, chlorpheniramine, carbinoxamine, and/or any salts thereof.

13. The method of claim 10, wherein said daily intake of N-nitrosodimethylamine administered to said patient in need of treatment is less than 40 ng per day when the composition is administered at the maximum daily dose per the respective drug label.

14. A method for inhibiting the formation of N-nitrosodimethylamine in a pharmaceutical composition, the method comprising the acts of:
providing an active pharmaceutical ingredient, wherein said active pharmaceutical ingredient is selected from the group consisting of metformin, valsartan, irbesartan, losartan, sumatriptan, diltiazem, tetracycline, doxylamine, chlorpheniramine, carbinoxamine, and/or any salts thereof; and
adding non-antioxidant acidic ingredient as an inhibitor of N-nitrosodimethylamine to said active pharmaceutical ingredient to form a pharmaceutical composition.

15. The method of claim 14, wherein said non-antioxidant acidic ingredient is selected from the group comprising hydrochloric acid, sulfuric acid, phosphoric acid, boric acid, benzene sulphonic acid, acetic acid, adipic acid, alginic acid, benzoic acid, carbomer, citric acid, fumaric acid, lactic acid, maleic acid, malic acid, tartaric acid, succinic acid, formic acid, oxalic acid, oleic acid, sorbic acid, pentetic acid, polycarbophil, salicylic acid, acetyl salicylic acid, glycine hydrochloride, L-cystine dihydrochloride, glutamic acid, aspartic acid, disodium edetate, edetic acid, salts of citrate monobasic, salts of phosphate monobasic, enteric polymers, or any combinations thereof.

16. The method of claim 14, further comprising the act of adding an excipient to form a pharmaceutical composition.

17. The method of claim 16, wherein said excipient is selected from the group comprising a binder, a filler, a lubricant, or a rate-controlling ingredient for dissolution of said active pharmaceutical ingredient.

18. The method of claim 14, wherein said pharmaceutical composition comprises less than 40 ng N-nitrosodimethylamine when the pharmaceutical composition is administered at the maximum daily dose per the respective drug label.

19. The method of claim 15, wherein said pharmaceutical composition comprises less than 40 ng N-nitrosodimethylamine when the pharmaceutical composition is administered at the maximum daily dose per the respective drug label.

20. The method of claim 16, wherein said pharmaceutical composition comprises less than 40 ng N-nitrosodimethylamine when the pharmaceutical composition is administered at the maximum daily dose per the respective drug label.
